(19) United States
(12) Patent Application Publication
(10) Pub. No.: US 2005/0135976 A1
(43) Pub. Date: Jun. 23, 2005

(54) Chemical dispensing apparatus

(76) Inventor: Chris L. Sagar, Bradenton, FL (US)

Correspondence Address: Arthur W. Fisher, III, Suite 316, 5553 West Waters Avenue, Tampa, FL, 33634, US

(21) Appl. No.: 10/743,579

(22) Filed: Dec. 22, 2003

(51) Int. Cl. A61L 9/14

(52) U.S. Cl. 422/124; 422/306

(57) ABSTRACT

A chemical dispensing apparatus for use with an air conditioning or heating system to reduce or eliminate airborne contaminates from the air stream of a conditioned space comprising a chemical supply section including a chemical reservoir and a chemical delivery device to store and supply chemical to the contaminated air stream and a dispensing control section including a microprocessor control to selectively control the dispensing of chemical to the contaminated air stream in response to at least one predetermined parameter.

### BACKGROUND OF THE INVENTION

1. Field of the Invention

An apparatus to dispense chemical for the purpose of reducing or eliminating odor causing microorganisms as well as mold, mildew and bacterial growth.

2. Description of the Prior Art

Typically, mold, mildew and other undesirable microorganisms are present throughout air conditioning and heating systems together with the conditioned space, depending on several different conditions that may be more prevalent at certain time than others.

Currently in the market and over several decades as recorded in numerous prior arts, such as U.S. Pat. No. 3,490,436, various air conditioning systems have been designed to selectively introduce water or agent, such as germicide, deodorant, medication into air circulated through the air conditioning or heating system at predetermined intervals of time for predetermined periods of time. For example: U.S. Pat. No. 5,957,771 shows aroma spraying mechanism utilizing pressurized dispenser; U.S. Pat. No. 4,601,886 discloses an air treatment apparatus utilizing interchangeable cassettes and distributed via an electrical pump and U.S. Pat. No. 3,974,941 discloses an automated aerosol mist dispenser utilizing a timer to introduce product. All of these prior arts directly dispense chemical or water at predetermined intervals of time with limiting control of the quantity of the dispensed fluid.

In contrast, the present invention utilizes a feedback mechanism to provide a means for monitoring operating conditions and controlling the amount of chemicals dispersed.

### SUMMARY OF THE INVENTION

The present invention relates to a chemical dispensing apparatus for use in combination with an air handler to reduce or eliminate airborne contaminants from a conditioned space comprising a chemical supply section and a dispensing control section.

The chemical supply section comprises a chemical reservoir and a chemical delivery section to selectively dispense a chemical into the air stream. The chemical delivery section comprises a chemical dispensing device coupled to the chemical reservoir through a chemical flow control to selectively control the flow of chemical from the chemical reservoir to the air handler.

The dispensing control section comprises a microprocessor control including a display to provide a visual display of the system status and a plurality of control keys or actuators to selectively control the frequency and duration of the chemical dispensing cycle.

When the chemical dispensing apparatus is in use, the contaminated air stream is drawn into the air handler where the chemical is injected. The treated air stream is then drawn through an evaporator and a blower to be discharged into the conditioned space.

The invention accordingly comprises the features of construction, combination of elements, and arrangement of parts which will be exemplified in the construction hereinafter set forth, and the scope of the invention will be indicated in the claims.

### BRIEF DESCRIPTION OF THE DRAWINGS

For a fuller understanding of the nature and object of the invention, reference should be had to the following detailed description taken in connection with the accompanying drawings in which:

FIG. 1 is a schematic diagram of the chemical dispensing apparatus of the present invention.

FIG. 2 is a schematic diagram of an alternate embodiment of the chemical dispensing apparatus of the present invention.

Similar reference characters refer to similar parts throughout the several views of the drawings.

### DETAILED DESCRIPTION OF THE PREFERRED EMBODIMENT

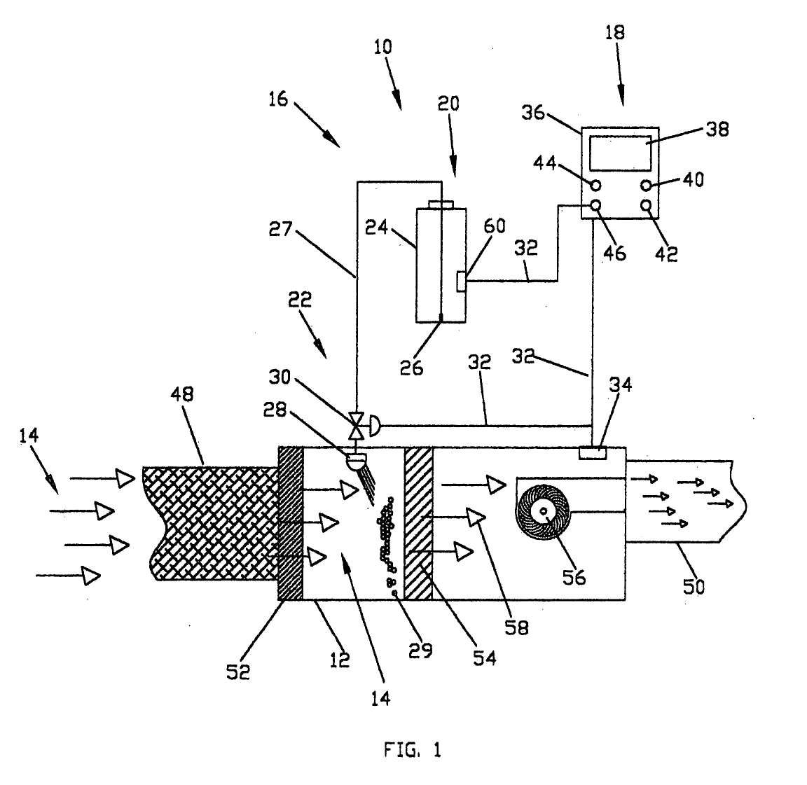

As shown in FIG. 1, the present invention relates to a chemical dispensing apparatus generally indicated as 10 for use in combination with an air handler 12 of an air conditioning or heating system to reduce or eliminate airborne contaminants from a contaminated air stream 14. The chemical dispensing apparatus 10 comprises a chemical supply section and a dispensing control section generally indicated as 16 and 18 respectively.

The chemical supply section 16 comprises a chemical reservoir generally indicated as 20 and a chemical delivery section generally indicated as 22. The chemical reservoir 20 comprises a chemical storage container 24 and a chemical feed control 26 such as a one-way check valve. The chemical delivery section 22 comprises a chemical dispensing device 28 such as an atomizing nozzle coupled to the chemical storage container 24 by a chemical supply conduit 27 through a chemical flow control 30 such as a normally closed flow control solenoid valve selectively movable between an open position and a closed position coupled to the dispensing control section 18 by a conductor 32 to receive control or actuating signals therefrom to selectively move from the normally closed position to the open position to allow the chemical 29 to flow from the chemical storage container 24 to the air handler 12 and a blower control 34 such as a 24 VAC transformer and blower control relay box by the conductor 32 to receive control or actuating signal therefrom to selectively actuate or energize a when the chemical 29 is dispensed from the chemical storage container 24.

The dispensing control section 18 comprises a microprocessor control device 36 including a display 38 to provide a visual display or indication of the system status, a program mode key 40 to select the desired operating mode of manual or automatic, a manual injection key 42 to manually dispense chemicals from the chemical storage container 24 into the air handler 12, and an up control key 44 and a down control key 46.

A return air conduit 48 and a supply air conduit 50 are operatively disposed at opposite end portions of the air handler 12; while, a filter 52, an evaporator 54 and the blower 34 are disposed in spaced relationship relative to each other within the air handler 12.

At initial set up of the microprocessor control device 36, the user will use chemical strips or a similar technique throughout the conditioned space to determine the amount of chemical 29 necessary to treat the entire conditioned space, to effectively reduce or eliminate odor causing microorganisms, as well as mold, mildew and bacterial growth. In addition, as each treatment application is initiated, the display 38 will display this information to user as well as any setup data programmed at initial start up.

The amount and application rate of the chemical 29 to be applied to the contaminated air stream 14 is determined at initial set up of the chemical dispersing apparatus 10 for the particular air-conditioning or heating system and conditioned space is programmed into the microprocessor control keys 46 or 48 and program mode key 40. The operating cycle is determined by the frequency of application and duration of dispensing of the chemical 29. Every time the air conditioning or heating system is energized or turned on through the air conditioning or heating system thermostat to condition the space, microprocessor control device 36 will monitor these actions through the conductor 32 and activate the blower 34.

When the chemical dispensing apparatus 10 is in use, the contaminated air stream 14 is drawn into the air handler 12 through the filter 52 by negative pressure created by the blower 56. When the contaminated air stream 14 with contaminants enters the air handler 12, the chemical 29 is injected into the contaminated air stream 14 under the control of the microprocessor control device 36. The treated air stream 58 is then drawn through the evaporator 54 into the blower 56 and discharged through the supply air conduit 50 into the conditioned space.

Specifically, as the contaminated air stream 14 is drawn into the air handler 12, the chemical 29 is injected into the contaminated air stream 14 by drawing the chemical 29 through the chemical feed control or check valve 26 through the chemical supply conduit 27 then through the control flow control or solenoid valve 30 in the open position and the chemical dispensing device 28 under the negative pressure in air handler 14 removing odor causing microorganisms, as well as mold, mildew and other bacteria. The chemically enriched conditioned air 56 entering the blower 56 is pressurized and fed through supply air conduit 50 into the conditioned space. Once entering the conditioned space the chemical 29 will be dispersed throughout entire conditioned space to further kill harmful microorganisms, mold, mildew and fungus.

The microprocessor control device 36 monitors and records the operation of the chemical dispensing apparatus 10 on a time or a minute-by-minute basis to accumulate data for a daily, weekly and monthly activity usage. This information will be used by microprocessor control device 36 to determine usage trends for different user operations for subsequent analysis to determine when chemical application is necessary. The microprocessor control device 36 also monitors consumption of the chemical 29 used either indirectly through rate calculations or directly through a level measurement sensor 60 and indicate on the display 38 the need to replenish the chemical storage container 24.

In addition, the chemical 29 may be manually dispensed by pressing the program mode key 40 and then pressing the manual injection key 42 to automatically apply proper amount as previously described. This occasional manual application information will be stored in data storage (not shown) within the microprocessor control device 36 for future reference in determining a predictive future application.

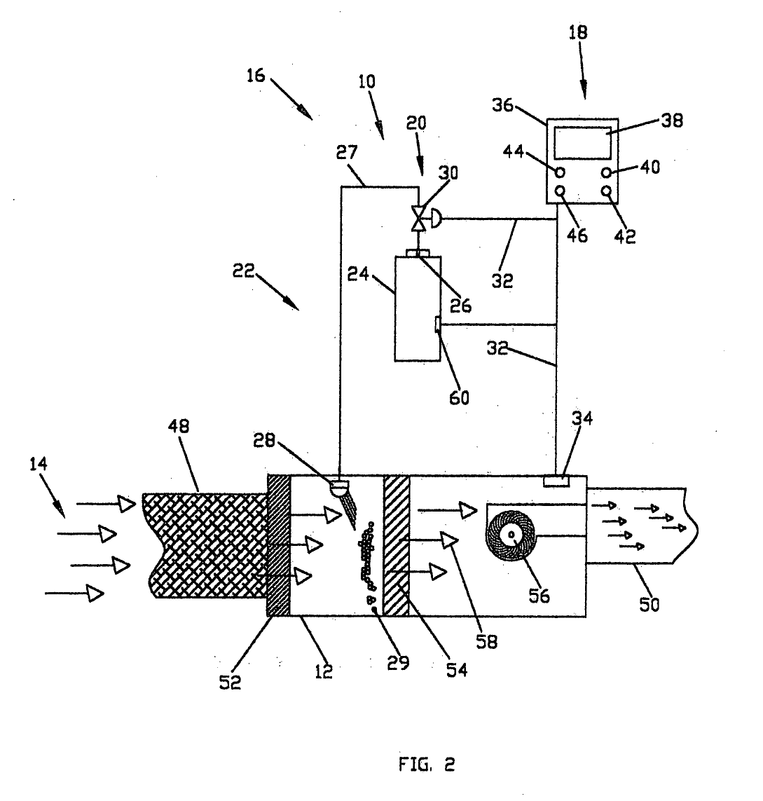

FIG. 2 shows shows an alternate embodiment of the chemical dispensing apparatus generally indicated as 10 for use in combination with an air handler 12 of an air conditioning or heating system to reduce or eliminate airborne contaminants from a contaminated air stream 14. As described hereinafter, the chemical reservoir 20 comprises a pressurized chemical storage container 22. Otherwise the invention is essentially the same. The chemical dispensing apparatus 10 comprises a chemical supply section and a dispensing control section generally indicated as 16 and 18 respectively.

The chemical supply section 16 comprises a chemical reservoir generally indicated as 20 and a chemical delivery section generally indicated as 22. The chemical reservoir 20 comprises a chemical storage container 24 such as an aerosol container and a chemical feed control 26 such as a nozzle. The chemical delivery section 22 comprises a chemical dispensing device 28 such as an atomizing nozzle coupled to the chemical storage container 24 by a chemical supply conduit 27 through a chemical flow control 30 such as a normally closed flow control solenoid valve selectively movable between an open position and a closed position coupled to the dispensing control section 18 by a conductor 32 to receive control or actuating signals therefrom to selectively move from the normally closed position to the open position to allow the chemical 29 to flow from the chemical storage container 24 to the air handler 12 and a blower control 34 such as a 24 VAC transformer and blower control relay box 34 by the conductor 32 to receive control or actuating signal therefrom to selectively actuate or energize a blower when the chemical 29 is dispensed from the chemical storage container 24.

The dispensing control section 18 comprises a microprocessor control device 36 including a display 38 to provide a visual display or indication of the system status, a program mode key 40 to select the desired operating mode of manual or automatic, a manual injection key 42 to manually dispense chemicals from the chemical storage container 24 into the air handler 12, and an up control key 44 and a down control key 46.

A return air conduit 48 and a supply air conduit 50 are operatively disposed at opposite end portions of the air handler 12; while, a filter 52, an evaporator 54 and the blower 34 are disposed in spaced relationship relative to each other within the air handler 12.

At initial set up of the microprocessor control device 36, the user will use chemical strips or a similar technique throughout the conditioned space to determine the amount of chemical 29 necessary to treat the entire conditioned space, to effectively reduce or eliminate odor causing microorganisms, as well as mold, mildew and bacterial growth. In addition, as each treatment application is initiated, the display 38 will display this information to user as well as any setup data programmed at initial start up.

The amount and application rate of the chemical 29 to be applied to the contaminated air stream 14 is determined at initial set up of the chemical dispersing apparatus 10 for the particular air-conditioning or heating system and conditioned space is programmed into the microprocessor control keys 46 or 48 and program mode key 40. The operating cycle is determined by the frequency of application and duration of dispensing of the chemical 29. Every time the air conditioning or heating system is energized or turned on through the air conditioning or heating system thermostat to condition the space, microprocessor control device 36 will monitor these actions through the conductor 32 and activate the blower 34.

When the chemical dispensing apparatus 10 is in use, the contaminated air stream 14 is drawn into the air handler 12 through the filter 52 by negative pressure created by the blower 56. When the contaminated air stream 14 with contaminants enters the air handler 12, the chemical 29 is injected into the contaminated air stream 14 under the control of the microprocessor control device 36. The treated air stream 58 is then drawn through the evaporator 54 into the blower 56 and discharged through the supply air conduit 50 into the conditioned space.

Specifically, as the contaminated air stream 14 is drawn into the air handler 12, the chemical 29 is injected into the contaminated air stream 14 by forcing the pressurized chemical 29 from the pressurized container 24 through the chemical feed control or nozzle 26 and the control flow control or solenoid valve 30 in the open position and the chemical dispensing device 28 through the chemical supply conduit 27 and chemical dispensing device 28 in to the air handler 14 odor causing microorganisms, as well as mold, mildew and other bacteria. The chemically enriched conditioned air 58 entering the blower 56 is pressurized and fed through supply air conduit 50 into the conditioned space. Once entering the conditioned space the chemical 29 will be dispersed throughout entire conditioned space to further kill harmful microorganisms, mold, mildew and fungus.

The microprocessor control device 36 monitors and records the operation of the chemical dispensing apparatus 10 on a time or a minute-by-minute basis to accumulate data for a daily, weekly and monthly activity usage. This information will be used by microprocessor control device 36 to determine usage trends for different user operations for subsequent analysis to determine when chemical application is necessary. The microprocessor control device 36 also monitors consumption of the chemical 29 used either indirectly through rate calculations or directly through a level measurement sensor 60 and indicate on the display 38 the need to replenish the chemical storage container 24.

In addition, the chemical 29 may be manually dispensed by pressing the program mode key 40 and then pressing key the manual injection 42 to automatically apply proper amount as previously described. This occasional manual application information will be stored in data storage (not shown) within the microprocessor control device 36 for future reference in determining a predictive future application.

The adaptive predictive or intuitive control will utilize a variation of a FIR (Finite impulse Response) digital filter technique. The FIR filtered response uses a sliding time window to provide a damping function or forgetting factor algorithm. The algorithm adjusts over time so that large transients caused by excessive use of the cleansing chemical at start-up or during unusual usage periods, due to weather or bacterial contamination, will not cause unusual behavior of the system. The system will be initially set up with a factory default setting which should provide adequate protection for 95% of the systems, based on tonnage of the HVAC system. This initial factory default value is based on system testing and calculated averages from various sized applications.

Therefore, it has been shown that the present invention provides new novel apparatus for applying chemical to eliminate odor causing microorganisms, as well as mold, mildew and bacterial growth, by utilizing intuitive microprocessor control chemical dispensing system.

Now that the invention has been described,

1. A chemical dispensing apparatus for use with an air conditioning or heating system to reduce airborne contaminates from the air stream of a conditioned space comprising a chemical supply section including a chemical reservoir and a chemical delivery device to store and supply chemical to the contaminated air stream and a dispensing control section including a microprocessor control to selectively control the dispensing of chemical to the contaminated air stream.

2. The chemical dispensing apparatus of claim 1 wherein said chemical reservoir comprises a chemical storage container and a chemical feed control.

3. The chemical dispensing apparatus of claim 2 wherein the chemical delivery section comprises a chemical dispensing device coupled to said chemical storage container by a chemical supply conduit through a chemical flow control.

4. The chemical dispensing apparatus of claim 3 wherein said chemical flow control comprises a normally closed flow control valve selectively movable between an open position and a closed position coupled to said dispensing control section by a conductor to receive actuating signals therefrom to selectively move from said normally closed position to said open position to allow the chemical to flow from said chemical storage container to the air handler.

5. The chemical dispensing apparatus of claim 4 wherein said chemical feed control comprises an atomizing nozzle coupled to the chemical storage container.

6. The chemical dispensing apparatus of claim 4 wherein said chemical feed control is a check valve.

7. The chemical dispensing apparatus of claim 6 further including a blower control to receive control or actuating signals to selectively actuate or energize a blower when chemical is dispensed from said chemical storage container.

8. The chemical dispensing apparatus of claim 7 wherein said blower control includes a transformer and blower control relay box.

9. The chemical dispensing apparatus of claim 1 wherein said dispensing control section comprises a microprocessor control device including a display to provide a visual display of the system status.

10. The chemical dispensing apparatus of claim 9 wherein said microprocessor control device controls the operating cycle by the frequency of application and duration of dispensing of the chemical.

11. The chemical dispensing apparatus of claim 10 wherein said microprocessor control device includes an up control key and a down control key to selectively control said operating cycle.

12. The chemical dispensing apparatus of claim 9 wherein said microprocessor control device monitors and records the operation of said chemical dispensing apparatus.

13. The chemical dispensing apparatus of claim 9 wherein said microprocessor control device monitors consumption of chemical.

14. The chemical dispensing apparatus of claim 13 wherein said microprocessor control devices monitors consumption of chemical through dispensing rate calculations.

15. The chemical dispensing apparatus of claim 14 wherein said display includes a display of chemical in said reservoir.

16. The chemical dispensing apparatus of claim 13 wherein said microprocessor control devices monitors consumption of chemical through a sensor.

17. The chemical dispensing apparatus of claim 16 wherein said display includes a display of chemical in said reservoir.

18. The chemical dispensing apparatus of claim 15 wherein the chemical may be manually dispensed by pressing a program mode key and then pressing a manual injection key.
